(19) United States
(12) Patent Application Publication
(10) Pub. No.: US 2026/0191574 A1
(43) Pub. Date: Jul. 9, 2026

(54) SYSTEM AND METHOD FOR VERTEBRAL BODY REPAIR

(71) Applicant: KIC VENTURES, LLC, BURLINGTON, MA (US)

(72) Inventors: KINGSLEY R. CHIN, BURLINGTON, MA (US); VITO LORE, BURLINGTON, MA (US); JOSHUA FINKEL-LOPEZ, BURLINGTON, MA (US)

(73) Assignee: KIC VENTURES, LLC, BURLINGTON, MA (US)

(21) Appl. No.: 19/556,405

(22) Filed: Mar. 4, 2026

Related U.S. Application Data
(63) Continuation-in-part of application No. 18/107,474, filed on Feb. 8, 2023.
(60) Provisional application No. 63/767,203, filed on Mar. 5, 2025.

(51) Int. Cl. A61B 17/88; A61B 34/20; A61L 27/10; A61L 27/22; A61L 27/36; A61L 27/54

(52) U.S. Cl. CPC A61B 17/8819; A61B 34/20; A61L 27/10; A61L 27/222; A61L 27/3695; A61L 27/54

(57) ABSTRACT

A method for inserting bone filler material into a defect of a vertebra includes providing a bone needle tool having an inner stylet component and an outer cannula component, and then inserting the bone needle tool into a pedicle and advancing the bone needle tool until a distal end of the inner stylet component pierces an endplate of the vertebra and reaches the defect. Next, removing the inner stylet component from the bone needle tool and attaching a syringe filled with bone filler material to a proximal end of the outer cannula component and then advancing a plunger of the syringe to deliver the bone filler material into the defect.

### CROSS REFERENCE TO RELATED CO-PENDING APPLICATIONS

This application claims the benefit of U.S. provisional application Ser. No. 63/767,203 filed Mar. 5, 2025 and entitled “SYSTEM AND METHOD FOR VERTEBRAL BODY REPAIR”, the contents of which are expressly incorporated herein by reference.

This application is a continuation-in-part and claims the benefit of U.S. non-provisional application Ser. No. 18/107,474 filed Feb. 8, 2023 and entitled “DEVICES AND METHODS FOR SPINAL FACET FUSION”, the contents of which are expressly incorporated herein by reference.

### FIELD OF THE INVENTION

The present invention relates to a system and a method for vertebral body repair, and more particularly to system and a method for treating defects formed in a vertebral body.

### BACKGROUND OF THE INVENTION

The human spine includes individual vertebras that are connected to each other. Under normal circumstances the structures that make up the spine function are configured to protect the neural structures, allow us to stand erect, bear axial loads, and are flexible for bending and rotation. Disorders of the spine occur when one or more of these spine structures are abnormal. In these pathologic circumstances, surgery may be tried to restore the spine to the normal state and to relieve the patient of pain. Spine surgery for a multitude of spinal disorders is often used for filling voids within a pathologic vertebral body (exemplified by kyphoplasty or vertebroplasty procedures), replacement of a degenerated intervertebral disc with an intervertebral implant device that preserves mobility (disc replacement) or for fusing adjacent vertebral segments (interbody and posterolateral fusions). In particular, devices, methods and materials are needed for treating defects within a pathologic vertebral body. Vertebral defects include voids (including Schmorl's nodes and voids formed post tumor removal), fractures, and modic changes in the vertebral endplates.

### SUMMARY OF THE INVENTION

The present invention relates to a system and a method for vertebral body repair, and more particularly to system and a method for treating defects that are formed in a vertebral body.

In general, in one aspect, the invention features a method for inserting bone filler material into a defect of a vertebra, including the following. First, providing a bone needle tool comprising an inner stylet component and an outer cannula component and inserting the bone needle tool into a pedicle and advancing the bone needle tool until a distal end of the inner stylet component pierces an endplate of the vertebra and reaches the defect. Next, removing the inner stylet component from the bone needle tool and attaching a syringe filled with bone filler material to a proximal end of the outer cannula component, and then advancing a plunger of the syringe to deliver the bone filler material into the defect.

Implementations of this aspect of the invention may include one or more of the following features. The bone filler material includes demineralized bone matrix (DBM), bioactive glass 45S5, and porcine gelatin. The bone filler material may be one of proteoglycans, nucleus pulposus cells, allograft bone, cadaveric endplate material, or combinations thereof. The defect is a Schmorl's node (SN) void and the bone filler material is inserted into the SN void. The bone needle tool is inserted using a lateral to medial and inferior to superior trajectory, in order to target a defect in a superior level vertebra. The bone needle tool is inserted using a lateral to medial and superior to inferior trajectory, in order to target a defect in an inferior level vertebra. The bone needle insertion, advancement and trajectory are guided via fluoroscopic imaging. The inner stylet component comprises an elongated rod having a handle attached to a proximal end and a distal end forming a sharp needle tip. The outer cannula component comprises an elongated cannula having a handle attached to a proximal end and a distal end having cutting threads formed on its outer surface. The elongated rod of the inner stylet component is sized to fit and slide within the elongated cannula of the outer cannula component, so that the sharp needle tip protrudes through a distal open end of the elongated cannula. The method of handle of the outer cannula component interlocks with the handle of the inner stylet component.

In general, in another aspect the invention features a system for inserting bone filler material into a defect of a vertebra, including a bone needle tool and a syringe. The bone needle tool includes an inner stylet component and an outer cannula component, and the syringe is filled with bone filler material. The bone needle tool is configured to be inserted into a pedicle and advanced until a distal end of the inner stylet component pierces an endplate of the vertebra and reaches the defect. The inner stylet component is configured to be removed from the bone needle tool and the syringe is configured to be attached to a proximal end of the outer cannula component. A plunger of the syringe is configured to be advanced to deliver the bone filler material into the defect.

### BRIEF DESCRIPTION OF THE DRAWINGS

Referring to the figures, wherein like numerals represent like parts throughout the several views:

FIG. 1A is a schematic side view of Schmorl's nodes (SN) in the lumbar vertebral region;

FIG. 1B depicts radiographic images of Schmorl's nodes (SN) in the lumbar vertebral region;

FIG. 2A-FIG. 2B depict perspective back and side views of the bone needle tool insertion into a pedicle to target a SN void in a superior level vertebra, respectively;

FIG. 2C-FIG. 2D depict perspective side and top views of an alternate trajectory for the bone needle tool insertion into a pedicle to target a SN void in an inferior level vertebra, respectively;

FIG. 3A-FIG. 3B depict perspective front and top views of the bone needle tool advancement through the pedicle until the needle tip pierces the vertebral endplate and protrudes into the center of the disc space to target a SN in a superior level vertebra;

FIG. 4A-FIG. 4B depict perspective side and top views of the bone needle tool advancement through the pedicle until the needle tip pierces the vertebral endplate and protrudes into the center of the disc space to target a SN void;

FIG. 5A-FIG. 5B depict perspective views of the connection and locking of a syringe to the bone needle tool;

FIG. 6A-FIG. 6B depict side and front perspective views of the insertion of the bioactive bone filler material via the syringe and the bone needle tool into the center of the disc space to fill the SN void, respectively;

FIG. 7A-FIG. 7B depict front and side perspective views of the lumbar vertebral region with the bioactive bone filler material filled SN void, respectively;

FIG. 9 depicts an alternate extra-pedicular trajectory for the bone needle tool to target a SN void in a vertebra;

FIG. 10A depicts a perspective view of the bone needle tool;

FIG. 10B depicts a perspective view of the inner stylet component of the bone needle tool of FIG. 10A; and

FIG. 11 is a flow diagram of the process for filling a void, such as a Schmorl's nod, in the vertebral body.

### DETAILED DESCRIPTION OF THE INVENTION

The present invention relates to a system and a method for vertebral body repair, and more particularly to system and a method for treating defects that are formed in a vertebral body.

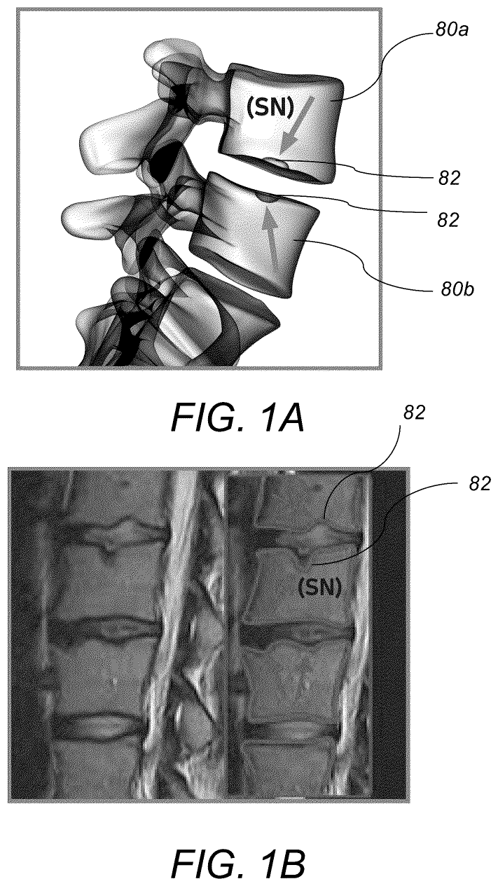

One specific type of vertebral pathology where filling of a void in the vertebral body is used is a Schmorl's node (SN). A Schmorl's node (SN) 82 is the herniation of nucleus pulposus (NP) through the cartilaginous and bony end plate into the body of adjacent vertebrae 80a, 80b, as shown in FIG. 1A and FIG. 1B. SNs are common findings on imaging, as shown in FIG. 1B, and although most SNs are asymptomatic, some have been shown to become painful lesions. In cases when the SN protrusions contact the marrow of a vertebra, they can cause inflammation and necrosis of the vertebral bone.

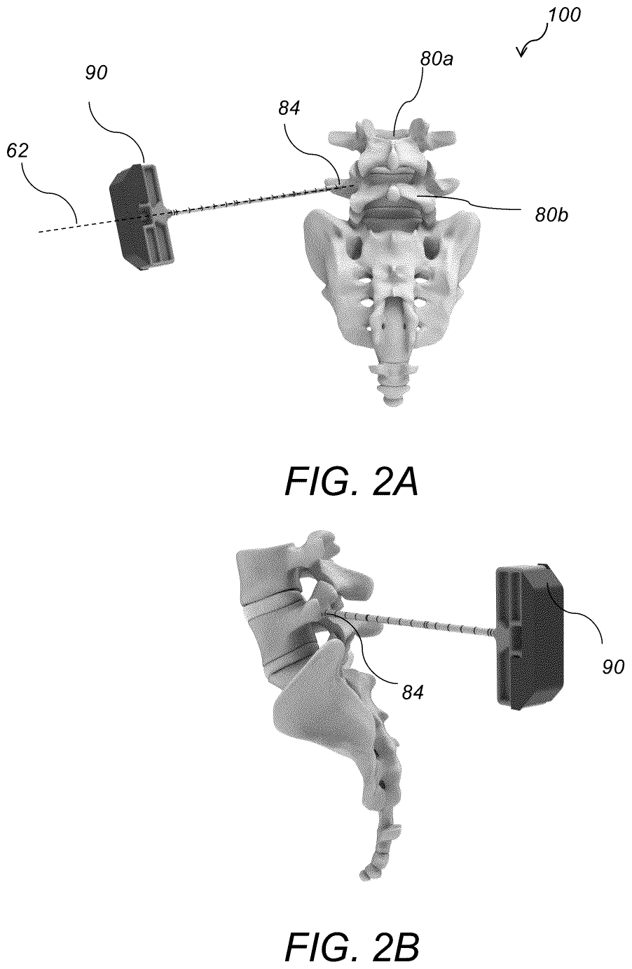

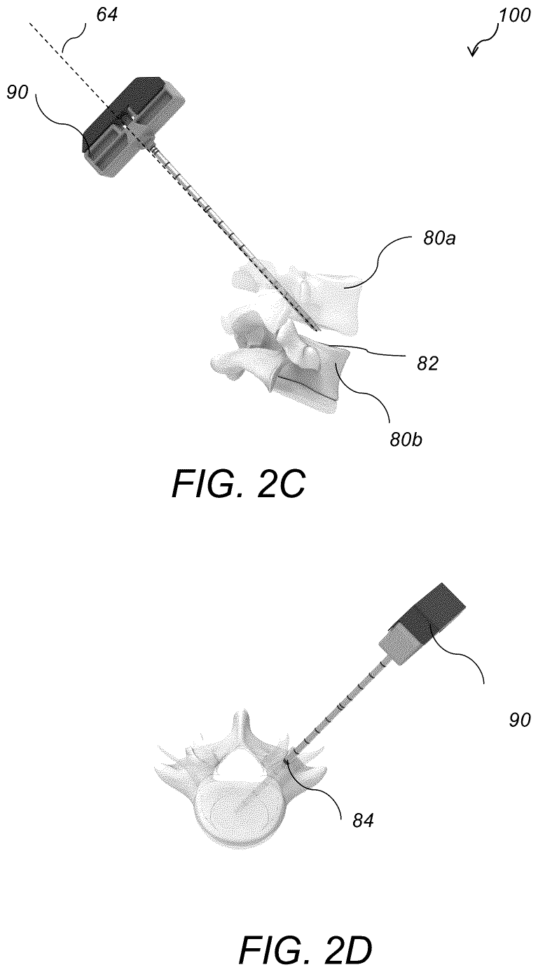

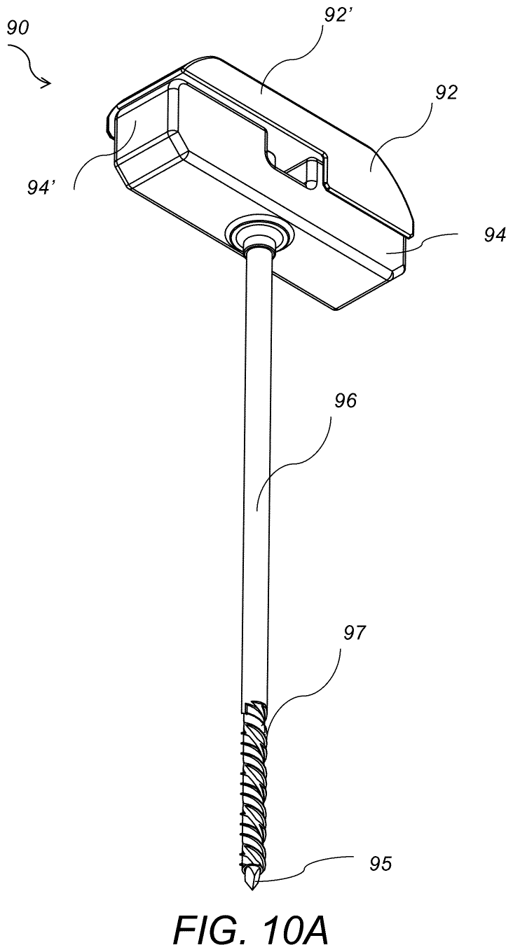

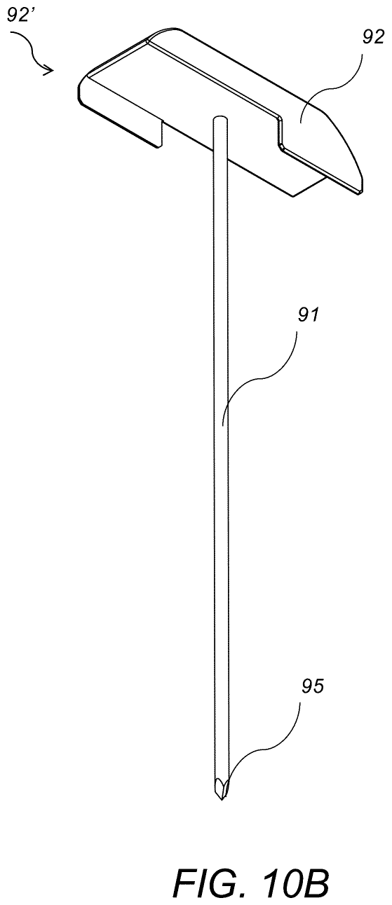

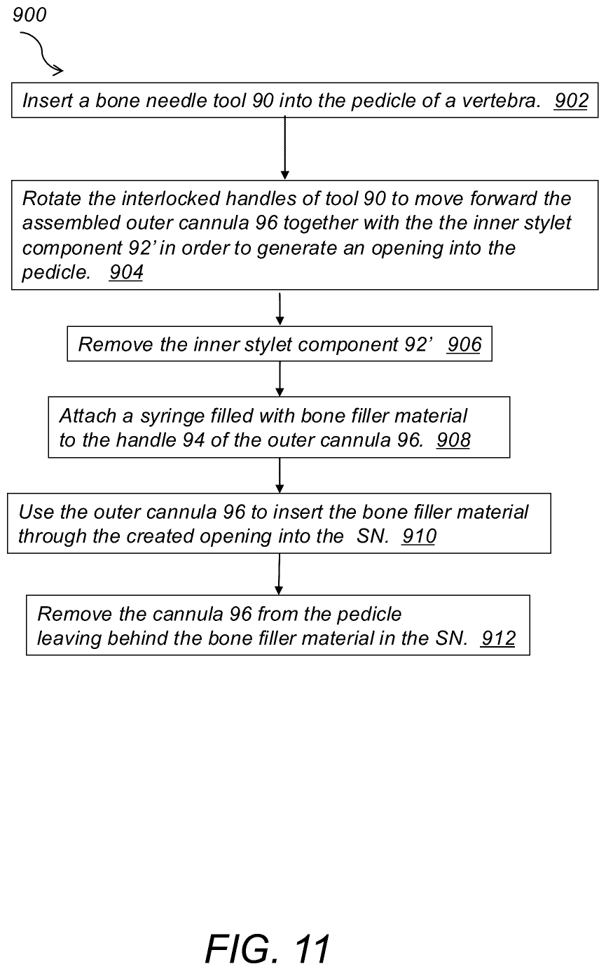

Referring to FIG. 2A-FIG. 7B and FIG. 11, the process 900 for filling a void, such as a Schmorl's node, in the vertebral body includes the following steps. First, a bone needle tool 90 is inserted into a pedicle 84 of a vertebra 80a (902). In one example, the bone needle tool 90 is described in the commonly owned U.S. patent application Ser. No.: 18/107,474, entitled “Devices and Methods for Spinal Facet Joint Fusion”, the contents of which are incorporated herein by reference. Referring to FIG. 10A and FIG. 10B, bone needle 90 includes an inner stylet component 92′ and an outer component 94′. Inner stylet component 92′ includes a handle 92 and an elongated rod 91. Elongated rod 91 has a proximal end attached to a bottom surface of the handle 92 and a distal end forming a trocar needle tip 95. The term trocar refers to a surgical instrument with a cutting needle tip. Outer component 94′ includes a handle 94 and an elongated cylindrical cannula 96. Elongated cannula 96 has a proximal end attached to a bottom surface of the handle 94 and a distal end 97 having cutting threads formed on its outer surface. The cutting threads may be single-lead threads, double-lead threads, triple-lead threads, quadruple-lead threads, or combinations thereof. Single-lead threads are formed by cutting one groove with a single-point tool. A double-lead thread has two grooves, a triple-lead thread has three grooves, and a quadruple-lead thread has four grooves. The lead is the distance that the cutting thread travels in one revolution. In one turn a single-lead cutting thread moves forward the distance (pitch) of one thread. Similarly, in one turn a double-lead cutting thread, moves by two threads, a triple-lead thread moves by three threads, and a quadruple-lead thread moves by four threads. In this embodiment, distal end 97 includes a combination of quad-lead cutting threads, and dual-lead cutting threads. The elongated rod 91 of the inner stylet component 92′ is sized to fit and slide within the elongated cannula 96 of the outer component 94′, so that the distal trocar needle tip 95 protrudes through the distal open end of the elongated cannula 96. The handle 92 of the inner stylet component 92′ sits onto and interlocks with the handle 94 of the outer component 94′. The two interlocked handles 92, 94 are used to rotate and move forward together the assembled cannula 90 with the trocar needle tip 95 of the inner stylet rod 91. The rotation of the interlocked handles 92, 94 causes the outer cutting threads 97 of the cannula 96 to create a thread profile in the pedicle and moves the trocar needle tip 95 of the inner stylet rod 91 forward to create an opening (904).

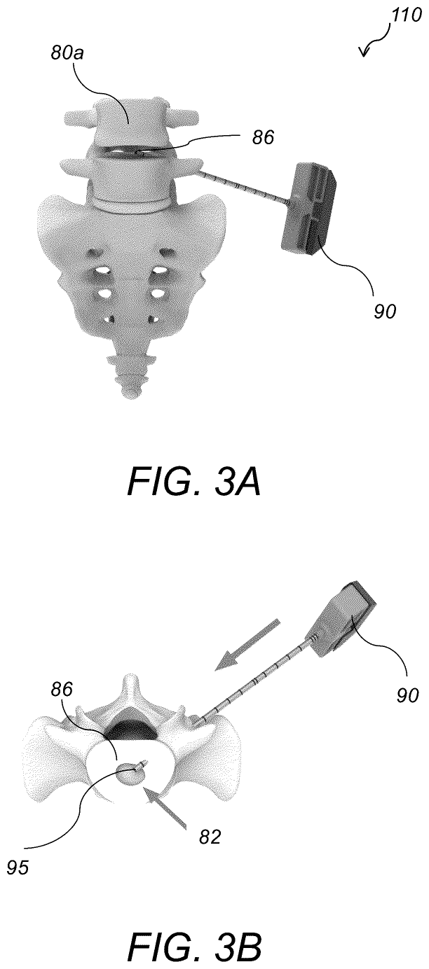

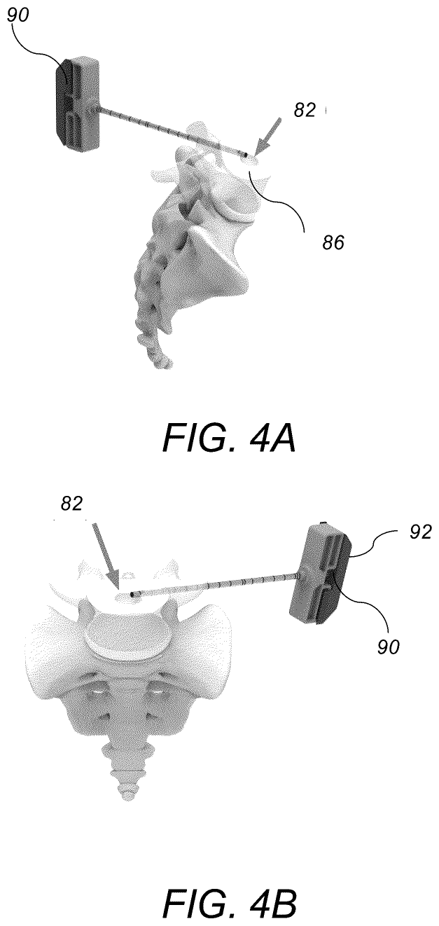

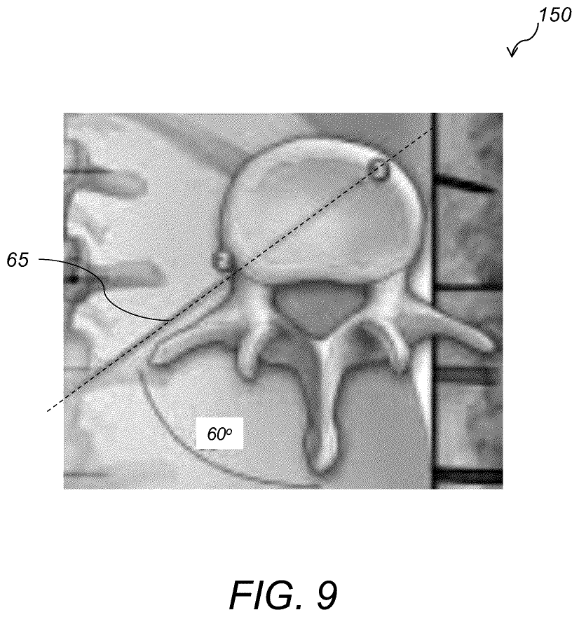

Referring back to FIG. 2A and FIG. 2B, the bone needle tool 90 is inserted using a lateral to medial and inferior to superior trajectory 62, in order to target a SN in a superior level vertebra 80a. The bone needle insertion, advancement and trajectory are guided via fluoroscopic imaging. The bone needle tool 90 tool is advanced until the needle tip 95 pierces the vertebral endplate and protrudes into the center of the disc space 86, as shown in FIG. 3A-FIG. 4B. The trajectory may be modified using fluoroscopy to ensure correct depth and trajectory. In other embodiments, an alternate trajectory is used targeting SNs 82 in an inferior vertebral level 80b, as shown in FIG. 2C-FIG. 2D. In this case, the bone needle is inserted into the pedicle using lateral to medial and superior to inferior trajectory 64, and the bone needle tool 90 tool is advanced until the needle tip 95 pierces the vertebral endplate and protrudes into the center of the disc space 86. In yet other embodiments, alternate trajectories 65 are used that include extra-pedicular trajectories, or through the vertebral body, as shown in FIG. 9.

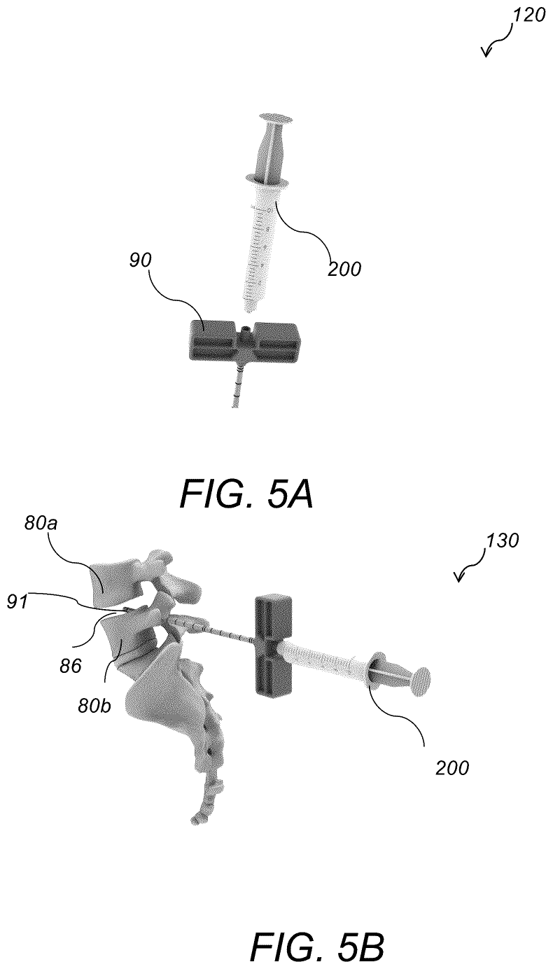

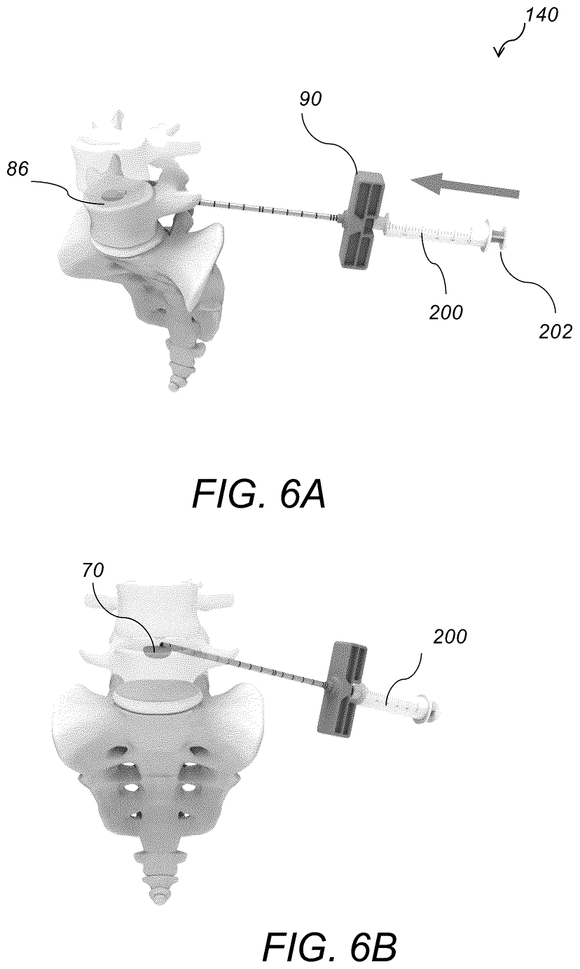

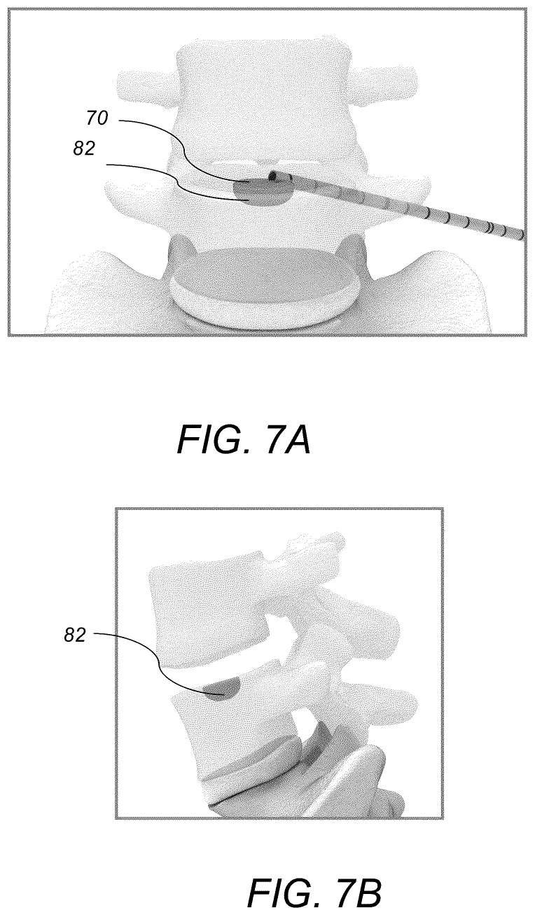

Next, the inner stylet component 92′ of the bone needle 90 is removed (906) and then a therapeutic syringe 200 is connected to the handle 94 of the bone needle 90 and rotated to lock in place, as shown in FIG. 5A and FIG. 5B (908). The therapeutic syringe 200 is filled with the bone filler material 70. In one example, the bone filler material 70 is a bioactive bone graft material. In the next step, the plunger 202 of the therapeutic syringe 200 is advanced to deliver the bone filler material 70 into the defect area to fill the SN void 82, as shown in FIG. 6A and FIG. 6B (910). The trajectory may be adjusted and additional bioactive bone filler material 70 may be introduced into the defect 82, as shown in FIG. 7A and FIG. 7B. Finally, the cannula 96 is removed leaving behind the bone filler material in the SN (912).

In one example, the bioactive bone graft material is a combination of the following materials combined in a matrix.
Demineralized Bone Matrix (DBM)—cadaveric
Bioactive Glass 45S5
Porcine Gelatin
   The product is sold under the tradename NanoFUSE® by NanoFUSE Biologics, LLC and is described in U.S. Pat. No. 7,846,459, the contents of which are incorporated herein by reference.

By injecting the bioactive bone filler material 70 into the SN voids 82, the vertebral bone defects are filled. Blood and fluid supply (i.e., saline) from the vertebral body are combined with constituent materials released from the NanoFUSE® and generate biologic activity of osteoblasts. In particular, when NanoFUSE® is mixed with an aqueous body fluid, such as blood or saline, it initiates the controlled release of calcium, sodium, silica, and phosphate ions. This ionic exchange creates a local environment favorable to cellular activity and hydroxyapatite formation. As the reaction progresses, a three-dimensional, ultra-porous calcium hydroxyapatite (HA) layer forms, providing an osteoconductive scaffold that supports new bone formation and remodeling. This NanoFuse® induced biologic activity of osteoblasts increases disc hydration and results in reversing disc degeneration.

In other examples, the bioactive bone filler material 70 is one of proteoglycans, nucleus pulposus cells, allograft bone, cadaveric endplate material, or combinations thereof.

Advantages of the present invention include one or more of the following. Unlike traditional approaches that penetrate the annulus and risk exacerbating disc degeneration, the present approach treats the disc indirectly through the vertebral endplate, preserving annular integrity. Fluoroscopy is used to ensure correct depth and trajectory. Unlike current solutions that rely on cadaveric nucleus pulposus cells, NanoFUSE®'s combination of DBM and nano-bioactive glass offers a more accessible and scalable solution. By injecting NanoFUSE® into the SN voids, the bone defect is filled and the open blood supply from the vertebral body is leveraged to increase disc hydration, potentially reversing disc degeneration.

Several embodiments of the present invention have been described. Nevertheless, it will be understood that various modifications may be made without departing from the spirit and scope of the invention. Accordingly, other embodiments are within the scope of the following claims.

What is claimed is:

1. A method for inserting bone filler material into a defect of a vertebra, comprising:
providing a bone needle tool comprising an inner stylet component and an outer cannula component;
inserting the bone needle tool into a pedicle and advancing the bone needle tool until a distal end of the inner stylet component pierces an endplate of the vertebra and reaches the defect;
removing the inner stylet component from the bone needle tool and attaching a syringe filled with bone filler material to a proximal end of the outer cannula component;
advancing a plunger of the syringe to deliver the bone filler material into the defect.

2. The method of claim 1, wherein the bone filler material comprises demineralized bone matrix (DBM), bioactive glass 45S5, and porcine gelatin.

3. The method of claim 1, wherein the bone filler material comprises one of proteoglycans, nucleus pulposus cells, allograft bone, cadaveric endplate material, or combinations thereof.

4. The method of claim 1, wherein the defect is a Schmorl's node (SN) void and the bone filler material is inserted into the Schmorl's node (SN) void.

5. The method of claim 1, wherein the bone needle tool is inserted using a lateral to medial and inferior to superior trajectory, in order to target a defect in a superior level vertebra.

6. The method of claim 1, wherein the bone needle tool is inserted using a lateral to medial and superior to inferior trajectory, in order to target a defect in an inferior level vertebra.

7. The method of claim 1, wherein the bone needle insertion, advancement and trajectory are guided via fluoroscopic imaging.

8. The method of claim 1, wherein the inner stylet component comprises an elongated rod having a handle attached to a proximal end and a distal end forming a sharp needle tip.

9. The method of claim 8, wherein the outer cannula component comprises an elongated cannula having a handle attached to a proximal end and a distal end having cutting threads formed on its outer surface.

10. The method of claim 9, wherein the elongated rod of the inner stylet component is sized to fit and slide within the elongated cannula of the outer cannula component, so that the sharp needle tip protrudes through a distal open end of the elongated cannula.

11. The method of claim 9, wherein the handle of the outer cannula component interlocks with the handle of the inner stylet component.

12. A system for inserting bone filler material into a defect of a vertebra, comprising:
a bone needle tool comprising an inner stylet component and an outer cannula component;
a syringe filled with bone filler material;
wherein the bone needle tool is configured to be inserted into a pedicle and advanced until a distal end of the inner stylet component pierces an endplate of the vertebra and reaches the defect;
wherein the inner stylet component is configured to be removed from the bone needle tool and the syringe is configured to be attached to a proximal end of the outer cannula component; and
wherein a plunger of the syringe is configured to be advanced to deliver the bone filler material into the defect.

13. The system of claim 12, wherein the bone filler material comprises demineralized bone matrix (DBM), bioactive glass 45S5, and porcine gelatin.

14. The system of claim 12, wherein the bone filler material comprises one of proteoglycans, nucleus pulposus cells, allograft bone, cadaveric endplate material, or combinations thereof.

15. The system of claim 12, wherein the defect is Schmorl's node (SN) void and the bone filler material is configured to be inserted into the SN void.

16. The system of claim 12, wherein the bone needle tool is configured to be inserted using a lateral to medial and inferior to superior trajectory, in order to target a defect in a superior level vertebra.

17. The system of claim 12, wherein the bone needle tool is configured to be inserted using a lateral to medial and superior to inferior trajectory, in order to target a defect in an inferior level vertebra.

18. The system of claim 12, wherein the bone needle insertion, advancement and trajectory are guided via fluoroscopic imaging.

19. The system of claim 12, wherein the inner stylet component comprises an elongated rod having a handle attached to a proximal end and a distal end forming a sharp needle tip.

20. The system of claim 19, wherein the outer cannula component comprises an elongated cannula having a handle attached to a proximal end and a distal end having cutting threads formed on its outer surface.

21. The system of claim 20, wherein the elongated rod of the inner stylet component is sized to fit and slide within the elongated cannula of the outer cannula component, so that the sharp needle tip protrudes through a distal open end of the elongated cannula.

22. The system of claim 20, wherein the handle of the outer cannula component interlocks with the handle of the inner stylet component.
